(19) United States
(12) Patent Application Publication
(10) Pub. No.: US 2023/0165810 A1
(43) Pub. Date: Jun. 1, 2023

(54) FENFLURAMINE FOR TREATMENT OF CONDITIONS ASSOCIATED WITH SPREADING DEPOLARIZATION

(71) Applicants: ZOGENIX INTERNATIONAL LIMITED, Berkshire (GB); BAYLOR COLLEGE OF MEDICINE, Houston, TX (US)

(72) Inventors: Bradley S. GALER, West Chester, PA (US); Stephen J. FARR, Orinda, CA (US); Thaddeus Cromwell REEDER, San Carlos, CA (US); Jeff NOEBELS, Houston, TX (US)

(73) Assignees: ZOGENIX INTERNATIONAL LIMITED, Berkshire (GB); BAYLOR COLLEGE OF MEDICINE, Houston, TX (US)

(21) Appl. No.: 17/992,254

(22) Filed: Nov. 22, 2022

Related U.S. Application Data
(60) Provisional application No. 63/285,027, filed on Dec. 1, 2021.

(51) Int. Cl. A61K 31/13; A61B 5/369

(52) U.S. Cl. CPC A61K 31/13; A61B 5/369

(57) ABSTRACT

Provided are methods of inhibiting spreading depolarization in a subject by administering a sigma-1 agonist or a sigma-1 positive modulator to the subject. In some cases the subject is administered fenfluramine as the sigma-1 positive modulator. In some cases, the subject has been diagnosed with epilepsy, traumatic brain injury, migraines, stroke, ischemic attacks, hypoxia or an increased risk of sudden unexpected death in epilepsy (SUDEP), or a combination thereof.

### FIELD OF THE INVENTION

The invention relates to the treatment of diseases or conditions associated with spreading depolarization, such as cortical spreading depolarization. More specifically, the invention relates to the use of fenfluramine for the treatment of such diseases and conditions that are associated with cortical spreading depolarization.

### INTRODUCTION

Broadly, spreading depolarization (SD) is a spreading loss of ion homeostasis, altered vascular response, change in synaptic architecture, and subsequent depolarization in electrical activity following an inciting neurological injury or an electrical activity aberration, such as seizures. The spreading depolarization can occur in different regions of the nervous system. For instance, spreading depolarization in the cerebral cortex is referred to as cortical spreading depolarization (CSD). Spreading depolarization can also occur in the brainstem, which can sometimes lead to Sudden Unexpected Death in Epilepsy (SUDEP) since the brainstem helps regulate heart rate and breathing. Spreading depolarization is also referred to as spreading depression.

### SUMMARY

It has been found that sigma-1 receptor agonists or positive modulators are useful in slowing or stopping the propagation of the spreading depolarization wave. Provided are methods of inhibiting spreading depolarization in a subject by administering a sigma-1 agonist or a sigma-1 positive modulator to the subject. In some cases the subject is administered fenfluramine as the sigma-1 positive modulator. In some cases, the subject has been diagnosed with epilepsy, traumatic brain injury, migraines, stroke, ischemic attacks, hypoxia or an increased risk of sudden unexpected death in epilepsy (SUDEP), or a combination thereof.

The present disclosure features the use of a sigma-1 receptor agonists or positive modulators, including positive allosteric and positive non-allosteric modulators, or pharmaceutically acceptable salts thereof in an amount effective to reduce or stop spreading depolarization, in the brain, particularly in the brain cortex. Spreading depolarization has been implicated in sudden death in epilepsy (SUDEP) when it occurs in the brainstem, an area of the brain responsible for maintaining cardiorespiratory rhythms. A previous disclosure in US 2021/0330610 has claimed the use of fenfluramine in reducing the risk or prevention of SUDEP through stimulation of 5-HT4 receptors by fenfluramine.

Cortical spreading depolarization (CSD) is characterized as a wave of electrophysiological hyperactivity followed by a wave of inhibition in the cerebral cortex. Cortical spreading depolarization describes a phenomenon that can involve depolarization waves of the neurons and neuroglia that propagates across the cortex at a velocity of about 1.5-9.5 mm/min. Increased glutamatergic activity is thought to be a factor in generating CSD. CSD can be induced by hypoxic conditions and facilitates neuronal death in energy-compromised tissue. CSD has also been implicated in migraine aura, where CSD is assumed to ascend in well-nourished tissue and is typically benign in most of the cases. Spreading depolarization within brainstem tissues regulating functions crucial for breathing and has been implicated in sudden unexpected death in epilepsy, by way of ion channel mutations such as those strongly linked with Dravet syndrome, a severe childhood epilepsy that appears to carry an unusually high risk of SUDEP.

Fenfluramine is a non-psychoactive amphetamine derivative drug that was once widely prescribed as an appetite suppressant to treat obesity. Fenfluramine is devoid of the psychomotor stimulant and abuse potential of D-amphetamine and interacts with certain 5-hydroxytryptamine (serotonin, 5-HT) receptors and the serotonin transporter to release 5-HT from neurons. Low dose fenfluramine has been shown to provide anticonvulsant/antiseizure activity in the treatment of Dravet Syndrome, previously known as severe myoclonic epilepsy in infancy or SMEI, and Lennox Gastaut syndrome, both rare and malignant epileptic syndromes. Fenfluramine is approved for use in the treatment of Dravet syndrome in the United States and parts of the European Union. Fenfluramine was first approved for use in treatment of obesity in the 1970s, and later combined with phentermine off-label to increase weight loss. Fenfluramine was removed from the market worldwide when it was found that with chronic use at doses of 60 to 120 mg it induced, in some patients, cardiac valve abnormalities or pulmonary hypertension.

Fenfluramine is also referred to as 3-trifluoromethyl-N-ethylamphetamine and has the structure:

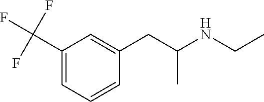 [SMILES: CCNC(C)Cc1cccc(C(F)(F)F)c1]

Systematic nomenclature for racemic fenfluramine is (RS)—N-ethyl-1-[3-(trifluoromethyl) phenyl]propan-2-amine or N-ethyl-alpha-methyl-3-(trifluoromethyl) benzeneethanamine. Fenfluramine readily forms acid addition salts, including pharmaceutically acceptable salts. Dexfenfluramine or (+) fenfluramine is the (S) enantiomer of fenfluramine. In functional assays, fenfluramine potentiated a (+)-SKF-10,047 [(+)N-allylnormetazocine]-induced increase in the twitch contraction amplitude and the Sig1 receptor [Sig1R]/binding immunoglobulin protein (BiP) dissociation induced by the Sig1R agonist PRE-084 [2-(morpholin-4-yl)ethyl 1-phenylcyclohexane-1-carboxylate], suggesting a positive modulatory action at Sig1R. [Maurice, T., et al. (2018). Fenfluramine is a sigma-1 receptor positive modulator in mice. Soc. Neurosci. Abstr. 692].

Other sigma-1 receptor (Sig1R) agonists and positive modulators include: Amitriptyline; Captodiame; Cocaine; Dextromethorphan; Dimethyltryptamine; Fluvoxamine; Hydrocodone; Noscapine; Pentazocine; Pentoxyverine (also called carbepentane, 2-[2-(diethylamino)ethoxy]ethyl 1-phenylcyclopentane-1-carboxylate); Pimavanserin; Prosterone; Remoxipride; PD 144418; 4-PPBP; Pentazocine; (+)-SKF 10,047; PRE-084; Pregnenolone sulfate (PREGS); carbetapentane; Dehydroepiandrosterone sulfate (DHEAS); SA 4503; Fambotizole; Fluvoxamine; AGY-94806; AE-37 (tetrahydro-N,N-dimethyl-2,2-diphenyl-3-furanmethanamine); igmesine; SKF 83959; MDMA; Memantine; Dehydroepiandro-sterone (DHES); SOMCL-668 and LS-1-137

Positive modulators of Sig1R and positive allosteric modulators of sigma-1 receptor (Sig1R) are described as compounds that can increase the activity of some Sig1R ligands that compete with (+)-pentazocine, one of the classic prototypical ligands that binds to the orthosteric Sig1R binding site. Non-limiting examples of positive modulators of Sig1R are: SKF83959; SKF38393; SCH23390; fenfluramine, methylphenylpiracetam and E1R (the 4R,5S-isomer of methylphenylpiracetam); Anavex 2-73; OZP002 [(±)-2-(3-chlorophenyl)-3,3,5,5-tetramethyl-2-oxo-[1,4,2]-oxazaphosphinane]. The two R-configuration enantiomers at position 4 of methylphenylpiracetam, (systematic chemical nomenclature is 2-(5-Methyl-2-oxo-4-phenyl-pyrrolidin-1-yl)-acetamide), i.e. (4R,5S) and (4R,5R), are the more active positive modulators of the sigma-1 receptor.

Other sigma-1 positive modulators have been disclosed in, for example, US 2013/0102571. Some sigma modulators are active at both sigma-1 and sigma-2 such as, for example DTG (1,3-Di-O-Tolylguanidine). In a preferred embodiment the compound of the invention is a Sig-1R agonist compound wherein said compound is an agonist selective for Sig-1R over Sig-2R (sigma-2 receptor), i.e. the compound is a selective Sig1R agonist. A compound is selective for Sig-1R over Sig-2R if it has a higher affinity for Sig-1R than Sig-2R, preferably an at least 5 times higher or at least 20 times higher or at least 50 times higher or, preferably, at least 102 higher or at least 103 higher or at least 104 higher affinity.

Sig1R is an endoplasmic reticulum membrane protein that, in addition to its promiscuous high-affinity ligand binding, has been shown to have chaperone activity. Different experimental approaches have been used to describe and validate the activity of allosteric modulators of Sig1R. Sig1R is an integral membrane-bound protein that is found in the nuclear membrane and endoplasmic reticulum and mitochondria-associated membrane (Mori et al., 2013; Mavlyutov et al., 2015; Su et al., 2016). Sig1R is expressed in both the CNS and peripheral tissues (Su and Junien, 1994). Sig1R is widely distributed in the brain, and it concentrates in specific areas involved in memory, emotion and sensory and motor functions (Alonso et al., 2000; Cobos et al., 2008). Sig1R, as described by its functional nature, is a chaperone protein and a unique cell protein modulator [reviewed in (Su et al., 2016)] that can amplify or reduce the signaling initiated when interacting with target proteins (Hayashi and Su, 2007; Zamanillo et al., 2012; Rodriguez-Munoz et al., 2015). Therefore, Sig1R demonstrates properties that can be attributed to both chaperone proteins and receptors. However, the notion that allosteric modulators of Sig1R have been identified provides additional support for the “receptor” view of Sig1R interactions.

Cortical spreading depolarization (CSD) is a slow, propagating reversible wave of network silence, characterized first by a wave of electrophysiological hyperactivity followed by a wave of inhibition in the cerebral cortex. Spreading depolarization can involve mass depolarization of neurons and glia lasting a minute or more. It arises focally and migrates as a wave across gray matter at a velocity of about 1.5-9.5 mm/min. SD can be generated by a sudden increase in cell membrane permeability to small ions in neurons and glia. However, this neurogenic origin does not preclude SD being initiated by local vascular changes. Increased extracellular potassium and high glutamatergic activity are thought to be both factors in generating and a consequence of CSD. The brain can recover from CSD, with the recovery of neuronal function occurring in the range of minutes to hours. In some cases, there is little or no permanent injury from CSD, while in other cases, neurologic damage or even death may occur.

CSD can be induced by hypoxic conditions and facilitates neuronal death in energy-compromised tissue. CSD has also been implicated in migraine aura, where CSD is assumed to ascend in well-nourished tissue and is typically benign in most of the cases. In the context of Dravet syndrome, spreading depolarization within the brainstem, which has nuclei that regulate functions crucial for breathing, has been implicated in sudden unexpected death in epilepsy. Dravet syndrome is caused by ion channel mutations, principally in Nav1.1 encoded by SCN1A and appears to carry an increased risk of SUDEP.

### BRIEF DESCRIPTION OF THE DRAWINGS

FIG. 1 shows a plot of the intrinsic optical signal as well as camera images of the induction of cortical spreading depolarization in a brain slice elicited by incrementally increasing KCl concentration in the ACSF bath.

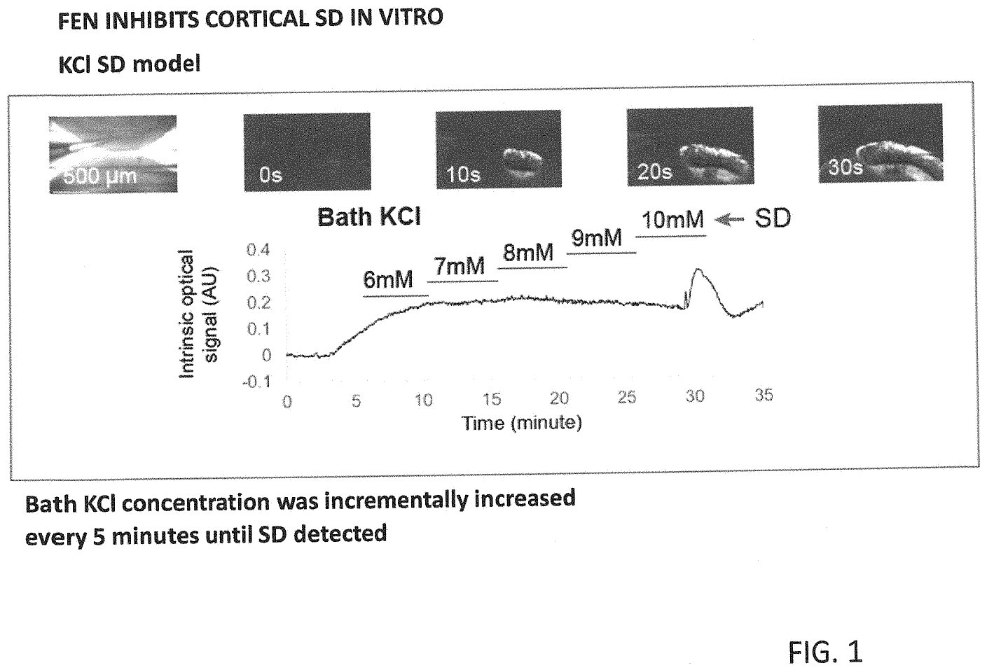

FIG. 2 shows a dose response relationship with increasing fenfluramine concentrations raising the KCl concentration necessary to initiate spreading depolarization.

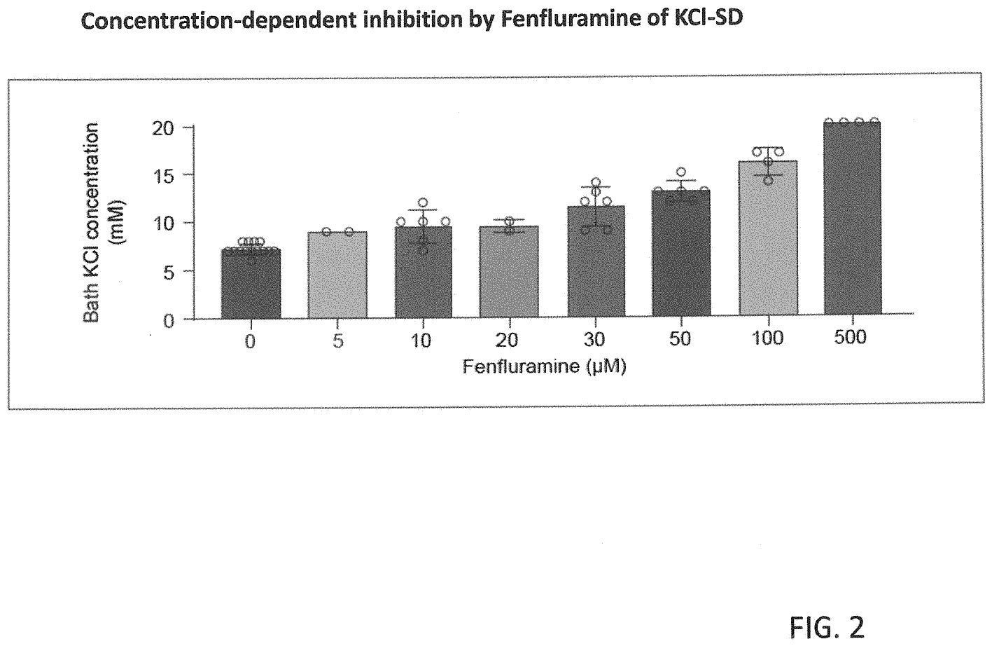

FIG. 3 shows a box plot of time to onset of spreading depolarization in an oxygen/glucose deprivation model with and without fenfluramine treatment. At 0% oxygen and 0 mM glucose there was a non-significant difference between treatment and non-treatment, while 0% oxygen and 2 mM glucose there was a significant increase in time to onset with treatment.

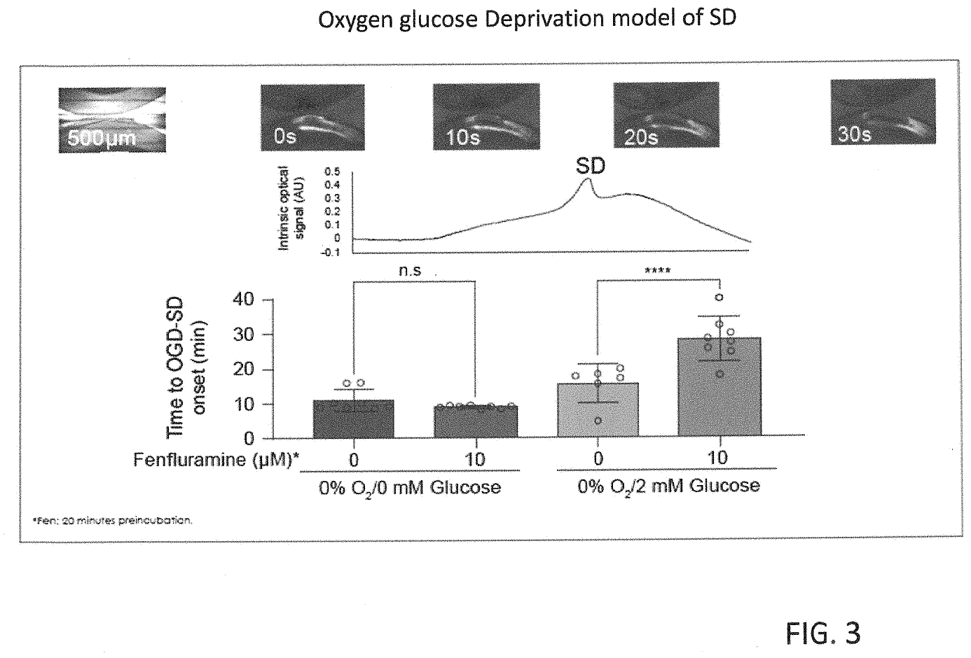

FIG. 4 shows two box plots 4A shows the effects of two sigma-1 agonists in raising the spreading depolarization threshold with treatment with dextromethorphan and carbepentane; while 4B shows a lack of additivity or synergism between fenfluramine and added serotonin.

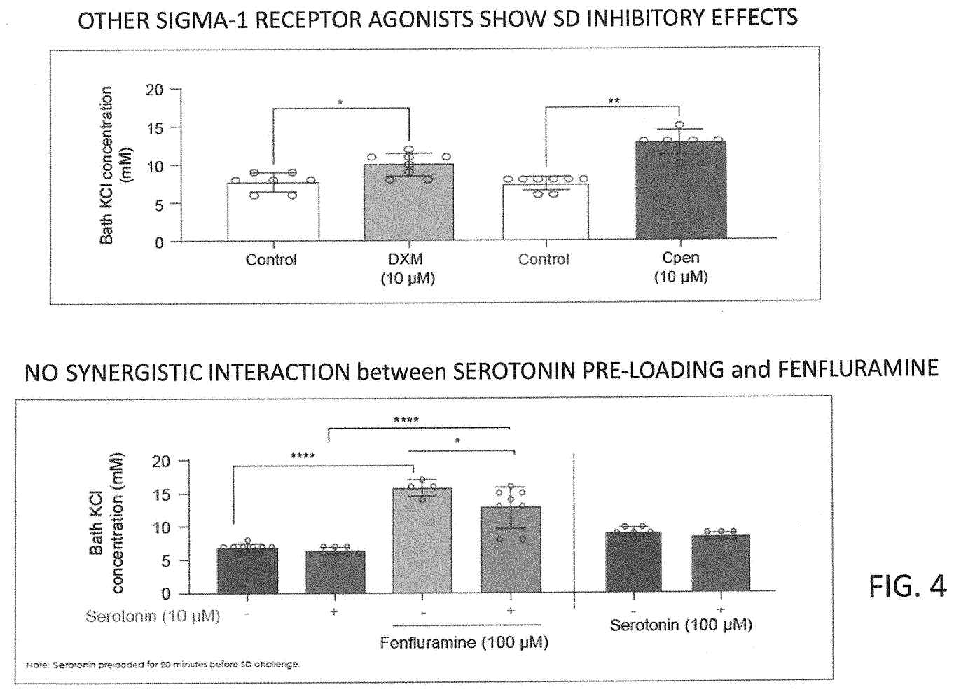

FIG. 5 shows that both serotonin and fenfluramine increase GABA spontaneous inhibitory postsynaptic currents, but blockade of the GABA receptors does not abolish fenfluramine's effects on spreading depolarization.

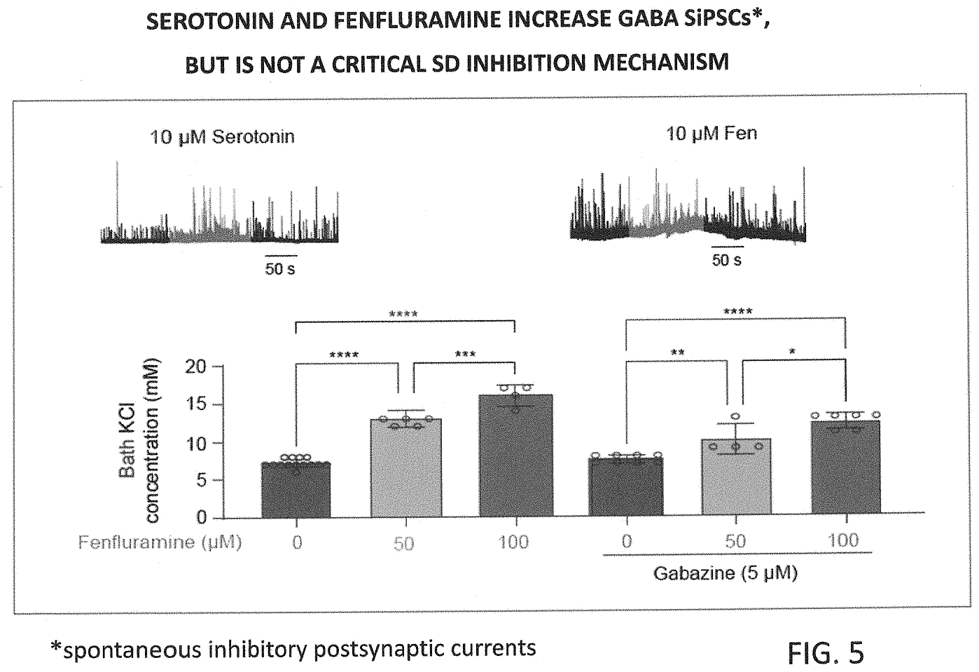

FIG. 6 shows that multi-5-HT receptor antagonist, asenapine, did not affect protective effects of fenfluramine in the KCl model of spreading depolarization, but did reduce spontaneous inhibitory postsynaptic currents of added serotonin in a separate experiment.

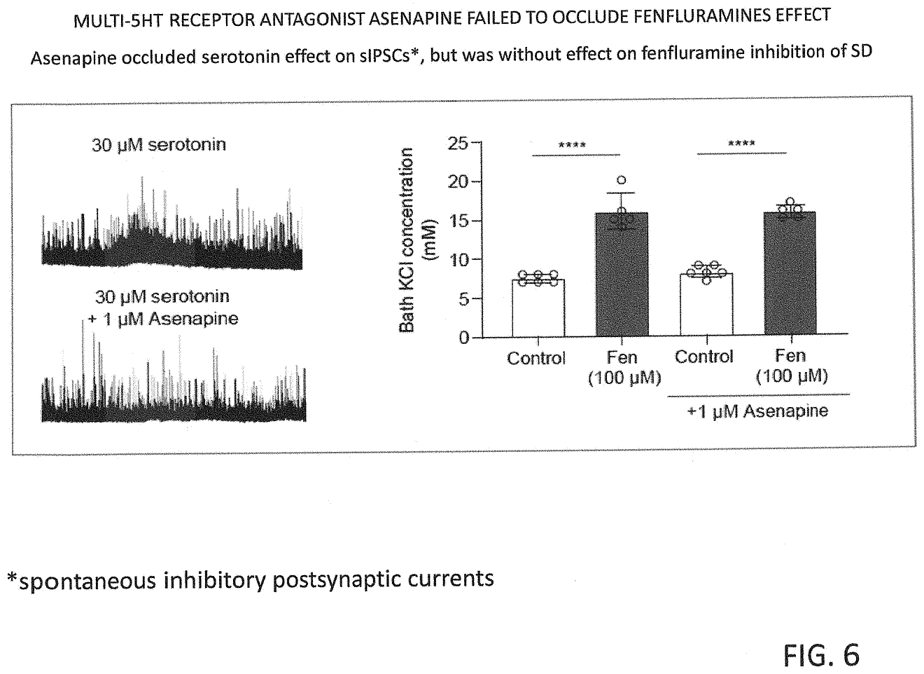

FIG. 7 shows that 5-HT4 receptor stimulation with a selective agonist does increase the threshold to spreading depolarization in the KCl model, but with the addition of a 5-HT4 antagonist to the fenfluramine experiment, the protective effect was only slightly diminished.

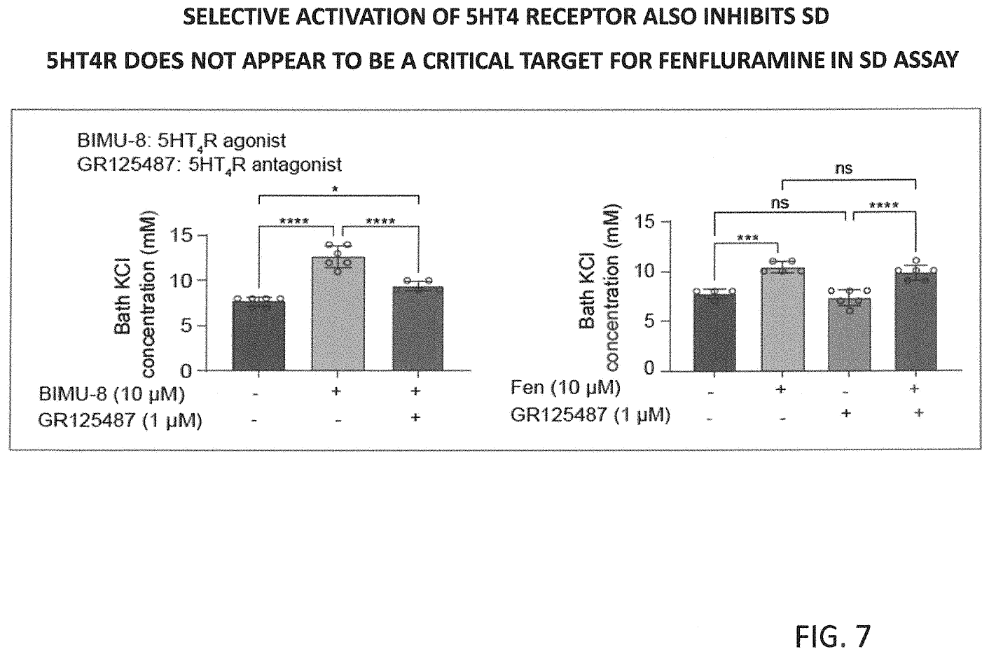

FIG. 8 shows a cartoon schematic summarizing the effect of positive and negative modulators on protein function.

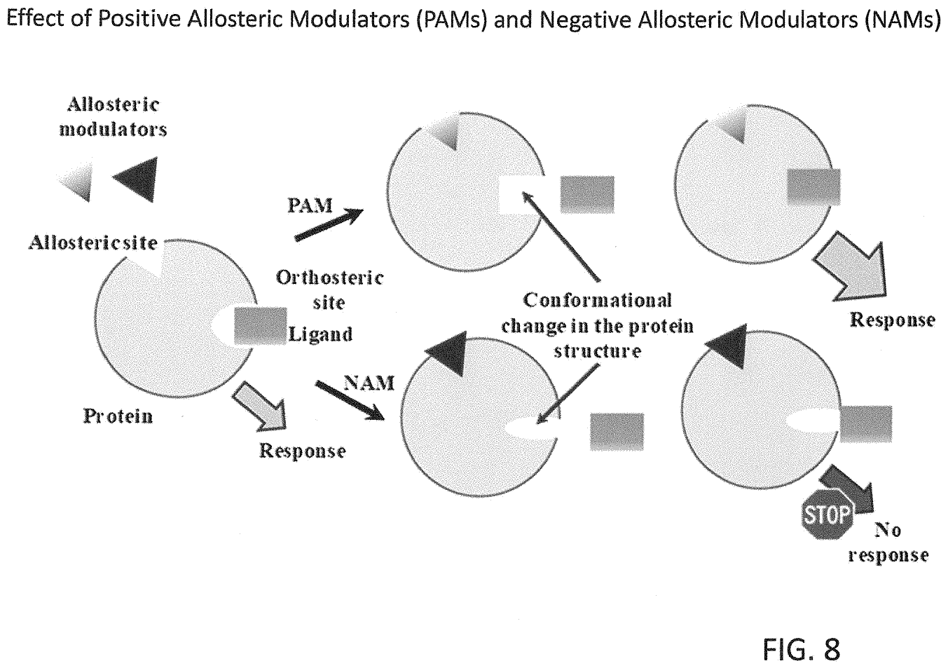

In all Figures where present: *p<0.05; **p<0.01; ***p<0.005; ****p<0.001 generated by the One-way ANOVA and post hoc Tukey's multiple comparisons test.

### DETAILED DESCRIPTION

Provided are methods of inhibiting spreading depolarization in a subject by administering a sigma-1 agonist or a sigma-1 positive modulator to the subject. In some cases the subject is administered fenfluramine as the sigma-1 positive modulator. In some cases, the subject has been diagnosed with epilepsy, traumatic brain injury, migraines, stroke, ischemic attacks, hypoxia or an increased risk of sudden unexpected death in epilepsy (SUDEP), or a combination thereof.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a droplet” includes a plurality of such droplets and reference to “the discrete entity” includes reference to one or more discrete entities, and so forth. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

### I. Abbreviations

Allosteric agonism, or positive allosteric modulation—means enhancement of protein function by direct or indirect mechanisms, including: binding and promoting a conformational change in the Sig1lR structure; regulation of Sig-lR activity through heteromeric protein-protein interactions; changing the ratio of monomers to oligomers of Sig-1R; changing the cell environment which activates SiglR (for example pH); or indirect regulation of SiglR through other proteins and signaling pathways.

Asenapine—an atypical antipsychotic medication with antagonist activity at many 5-HT receptor subtypes.

BIMU-8—3-isopropyl-N-[(1R,5S)-8-methyl-8-azabicyclo[3.2.1]octan-3-yl]-2-oxo-1,3-benzodiazole-1-carboxamide, a 5-HT4 receptor agonist

Cpen—carbepentane (also called pentoxyverine)

CSD—cortical spreading depolarization

DXM—dextromethorphan

Gabazine—A GABA receptors antagonist which blocks the actions of endogenous gamma-aminobutyric acid and GABA receptor agonists

GR125487—[1-[2-(methanesulfonamido)ethyl]piperidin-4-yl]methyl-5-fluoro-2-methoxy-1H-indole-3-carboxylate, a 5-HT4 receptor antagonist

KCl—potassium chloride

OGD—oxygen glucose deprivation

Positive modulator of Sigma-1R—

PRE-084—2-(4-Morpholino)ethyl-1-phenylcyclohexane-1-carboxylate

SCN1a+/− an organism having one null allele and one functional allele for the sodium channel 1 alpha subunit of the NaV1.1 sodium channel

SD—spreading depolarization

sIPSCs—spontaneous inhibitory postsynaptic currents

SKF83959—3-Methyl-6-chloro-2,3,4,5-tetrahydro-7,8-dihydroxy-1-(3-methylphenyl)-1H-3-benzazepine

SKF38393—2,3,4,5-Tetrahydro-7,8-dihydroxy-1-phenyl-1H-3-benzazepine

SCH23390—7-Chloro-3-methyl-1-phenyl-1,2,4,5-tetrahydro-3-benzazepin-8-ol

### II. Terms

Unless otherwise noted, technical terms are used according to conventional usage.

Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Cortical spreading depolarization (CSD) is used to describe the phenomenon of a slow, propagating reversible wave of network silence in all or parts of the cerebral cortex.

Hemorrhage: Bleeding or escape of blood from a vessel, including hemorrhagic stroke or burst aneurysms.

Hypoxia: The lack of oxygen supply to the tissues of the body below the normal level. Hypoxia can be caused by many different factors, which include drowning, smoke inhalation, strangulation, hemorrhagic or ischemic stroke, asthma, prolonged seizures epileptic seizures. Cerebral hypoxia refers to the brain not receiving or not being able to process enough oxygen. Premature infants often suffer cerebral hypoxia in the days and weeks after birth which is often associated with persistent motor (including cerebral palsy), sensory, and cognitive impairment.

Ischemia: A vascular phenomenon in which a decrease in the blood supply to a bodily organ, tissue, or part is caused, for instance, by constriction or obstruction of one or more blood vessels. Ischemia sometimes results from vasoconstriction, thrombosis or embolism. Ischemia can lead to direct ischemic injury, tissue damage due to cell death caused by reduced oxygen supply. In some cases, ischemia can lead to demyelination.

Migraine without Aura is the most common form of migraine. It presents as a headache that is usually on one side of the head and is often characterized by throbbing pain, which can be worsened by moving such as by walking or climbing the stairs. Its symptoms are so severe that the sufferer cannot do normal daily activities. Other symptoms include nausea or vomiting and sensitivity to light (photophobia), sound (phonophobia) and/or smells.

Hemiplegic Migraine causes a temporary weakness on one side of the body as part of their migraine attack (hemiplegia means paralysis on one side of the body).

The weakness may be in addition to some more common aura symptoms such as:

Visual disturbances—changes in eyesight in both eyes, such as colored spots, zigzags or sparkles;

Speech difficulties—slurring words or not being able to speak clearly;

Communication difficulties—the ability to write and understand language can be affected, causing problems with reading, listening, speaking and writing;

People may also experience:

dizziness or vertigo (a sensation of movement);

hearing problems or ringing in the ears;

confusion.

Hemiplegic migraine symptoms are similar to symptoms of a stroke. The weakness may last from one hour to several days, but usually subsides within 24 hours. A headache may follow the weakness, though it may occur before it or not at all. There are two types of hemiplegic migraine: familial and sporadic.

Familial hemiplegic migraine: Familial hemiplegic migraine, is the form that runs in families. When this occurs, at least two or more people in the same family experience weakness on one side of the body as a symptom with their migraine. On average 50% of children of a parent with hemiplegic migraine will develop the disorder. Three genes having various mutations are associated with familial hemiplegic migraine: CACNA1A, ATP1A2 and SCN1A.

The mutations cause ion channels in nerve cells to work incorrectly from time to time, resulting in a hemiplegic migraine attack. However, these gene specific mutations are not present in all families diagnosed with familial hemiplegic migraine. All SCN1A mutations reported in sporadic/familial HM3 are missense mutations and most of the experimental results show that they cause a gain of function of Na_{V} 1.1 as opposed to the loss of function of the epileptogenic Na_{V} 1.1 mutations. [Epilepsia 2019 December; 60 Suppl 3:S17-S24. doi: 10.1111/epi.16386.]

Sporadic hemiplegic migraine: Sporadic Hemiplegic Migraine or SHM is diagnosed when someone experiences all the physical symptoms of FHM but doesn't have a known family or inherited connection. The cause of SHM is unknown but probably due to new or ‘sporadic’ gene mutations. People with SHM usually also experience the more common aura symptoms with their attacks. For the most people the aura symptoms last around an hour to a day but can last longer.

Migraine with Aura The warning sign is most commonly a symptom that affects your sight, such as blind spots or seeing flashing lights. Auras can either happen on their own or together with the symptoms of a migraine without aura. The auras usually happen before a headache, which varies in severity and in some people does not happen. Auras typically start happening gradually over about five minutes and last for up to an hour. Auras most commonly affect sight, but speech can also be affected. Disorientation or confusion, or syncope may happen, although this is rare. Common symptoms related to sight include blind spots; seeing colored spots or lines; seeing flashing or flickering lights, seeing zig zag patterns; temporary blindness.

Other aura symptoms may include numbness or tingling sensation like pins and needles in parts of the body; muscle weakness; and feeling dizzy or off balance.

Migraine with brainstem aura, formerly called basilar-type migraine: symptoms often develop gradually and occur with or before a typical migraine headache in those who experience it. It occurs in about 1 in 10 people who get migraine with typical visual aura. Vertigo, dizziness, slurred speech, ringing in the ears and double vision would also commonly occur and some people experience disorientation or confusion as well as temporary loss of consciousness (syncope). Migraine with aura has a slightly higher risk of stroke than migraine without aura; however, there is no evidence that migraine with brainstem aura has a higher risk of stroke than migraine with typical aura.

Pharmaceutical composition: A composition containing fenfluramine, or a pharmaceutically acceptable salt thereof, formulated with a pharmaceutically acceptable excipient, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup) or a flexible-dosing, oral solution; for topical administration (e.g., as a cream, gel, lotion, or ointment); for intravenous administration (e.g., as a sterile solution free of particulate emboli and in a solvent system suitable for intravenous use); or in any other formulation described herein.

Pharmaceutically acceptable salt: A salt of fenfluramine which is, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of fenfluramine or separately by reacting the amine group with a suitable acid. and the like. For example, U.S. Pat. No. 10,351,509 describes a synthesis of fenfluramine and pharmaceutically acceptable salts thereof.

Pharmaceutically acceptable excipient (pharmaceutically acceptable carrier): Any ingredient other than fenfluramine, or a pharmaceutically acceptable salt thereof (e.g., a vehicle capable of suspending or dissolving the active compound) and having the properties of being nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

Preventing, treating or ameliorating a disease: “Preventing” refers to a prophylactic treatment or treatment that prevents one or more symptoms or conditions of a disease, disorder, or neuronal network condition described herein. Preventive treatment that includes administration of a sigma-1 agonist or sigma-1 positive modulator, or a pharmaceutically acceptable salt thereof, or a pharmaceutical composition thereof, can be acute, short-term, or chronic. The doses administered may be varied during the course of preventative treatment. “Treating” refers to an approach for obtaining beneficial or desired results, e.g., clinical results. Beneficial or desired results can include, but are not limited to, alleviation or amelioration of one or more symptoms or conditions; diminishment of extent of disease or condition; stabilized (i.e., not worsening) state of disease, disorder, or condition; preventing spread of disease or neuronal network condition; delay or slowing the progress of the disease or condition; amelioration or palliation of the disease or condition; and remission (whether partial or total), whether detectable or undetectable. “Ameliorating (palliating)” a disease or condition means that the extent and/or undesirable clinical manifestations of the disease, disorder, or condition are lessened and/or time course of the progression is slowed or lengthened, as compared to the extent or time course in the absence of treatment.

Subject: An animal (e.g., a mammal, such as a human). A subject to be treated according to the methods described herein may be one who has been diagnosed with a, or one at risk of developing the condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

Therapeutically effective amount: A quantity of a sigma-1 agonist or sigma-1 positive modulator or a pharmaceutically acceptable salt thereof, sufficient to achieve a desired effect in a subject, or in a cell, being treated with the agent. The effective amount of sigma-1 agonist or sigma-1 positive modulator depends on several factors, including, but not limited to the subject or cells being treated, and the manner of administration of the therapeutic composition. In some embodiments, a “therapeutically effective amount” of sigma-1 agonist or sigma-1 positive modulator, or a pharmaceutically acceptable salt thereof, is the amount sufficient to treat the disease state in a subject. In other embodiments, a “therapeutically effective amount” of a sigma-1 agonist or sigma-1 positive modulator, or a pharmaceutically acceptable salt thereof, is the amount sufficient to inhibit spreading depolarization, including cortical spreading depolarization in a subject.

### Methods

Provided are methods of inhibiting spreading depolarization in a subject by administering a sigma-1 agonist or a sigma-1 positive modulator to the subject, thereby inhibiting the spreading depolarization.

Provided are methods of treating a subject with a disease associated with spreading depolarization comprising: administering a therapeutically effective amount of a sigma-1 agonist or a sigma-1 positive modulator to the subject, and thereby alleviating or preventing symptoms or morbidity of the disease.

In some cases, the spreading depolarization is cortical spreading depolarization. In some cases the spreading depolarization is brainstem spreading depolarization.

As used herein, since the method comprises administering the sigma-1 agonist or sigma-1 positive modulator, the method as recited also includes cases wherein both a sigma-1 agonist is administered and a sigma-1 positive modulator is administered. The compounds are administered in an amount that is therapeutically effective for inhibiting the spreading depolarization. Exemplary routes of administration include oral, parenteral, intrathecal, or bolus injection. In some cases, the administration uses an intravenous drip. The terms sigma-1 agonist and sigma-1 positive modulator include the compounds themselves as well as pharmaceutically acceptable salts thereof.

Exemplary sigma-1 agonists include PRE-084, Blarcamesine, Donepezil, Fluvoxamine, Citalopram, Amitriptyline, L-687,384, Cutamesine, Dextromethorphan, N,N-Dimethyltryptamine, Pentazocine, and Opipramol and DTG.

In some cases, the subject is administered a sigma-1 positive modulator. Exemplary sigma-1 positive modulators include: SKF83959; SKF38393; SCH23390; fenfluramine, methylphenylpiracetam, E1R (the 4R,5S-isomer of methylphenylpiracetam); Anavex 2-73; OZP002 [(±)-2-(3-chlorophenyl)-3,3,5,5-tetramethyl-2-oxo-[1,4,2]-oxaza-phosphinane. In other cases, the sigma-1 agonist is fenfluramine. In other cases, the sigma-1 agonist is not fenfluramine. For example, the fenfluramine can be administered in an amount of 0.5 mg/kg/day to 5 mg/kg/day and wherein the dose is not greater than 120 mg/day.

In some embodiments the subject was diagnosed with spreading depolarization or an elevated risk of spreading depolarization before the administering. In some cases the subject was diagnosed with cortical spreading depolarization or brainstem spreading depolarization. In some cases, the diagnosing comprises performing an electroencephalography (EEG) measurement of the subject. See, for example, Drenckhahn C, et al. Correlates of spreading depolarization in human scalp electroencephalography. Brain (2012) 135:853-68. doi:10.1093/brain/aws010. For example, a first EEG can be performed, the subject can be administered a test amount of the sigma-1 agonist or sigma-1 positive modulator, and then performing a second EEG measurement. For example, if the EEG can indicate that administered the compound resulted in a reduction (e.g. disappearance) in one or more signals associated with cortical spreading depolarization. In some embodiments the EEG is performed with implanted electrodes. In some cases the administering is performed between 10 seconds and 10 days after the diagnosing, such as between 20 seconds and 1 day, between 25 seconds and 5 hours, between 30 seconds and 60 minutes, and between 1 minute and 30 minutes.

In some cases, the subject has been diagnosed with one or more conditions before the administering, e.g. conditions associated with spreading depolarization. In some cases the subject has been diagnosed with epilepsy, traumatic brain injury (TBI), migraines, migraines with aura, familial hemiplegic migraine, stroke, ischemia, oxygen deprivation, cerebral amyloid angiopathy, chronic subdural hematoma, or a combination thereof. In some embodiments the method includes diagnosing the subject with such conditions. In some cases, the subject is diagnosed with epilepsy, e.g. Dravet syndrome. In some cases, the subject has been diagnosed with an increased risk of sudden unexpected death in epilepsy (SUDEP), e.g. based on diagnosis of another condition such as epilepsy.

In some embodiments the method includes detecting SD via scalp EEG equipment programmed to measure a reduction or disappearance of cortical electrical signals or via implanted electrodes and administering a therapeutically effective amount of a sigma-1 agonist or sigma-1 positive modulator or pharmaceutically acceptable salt thereof to a subject

In some embodiments the method includes detecting SD via scalp EEG equipment programmed to measure a reduction or disappearance of cortical electrical signals or via implanted electrodes and fenfluramine or pharmaceutically acceptable salt thereof is administered at a dose of about 0.5 mg/kg/day to about 5 mg/kg/day and wherein the dose is not greater than 120 mg/day to a subject.

In some embodiments the method includes: detecting SD via scalp EEG equipment programmed to measure a reduction or disappearance of cortical electrical signals or via implanted electrodes and fenfluramine or pharmaceutically acceptable salt thereof is administered at a dose of about 0.1 mg/kg/day to about 2 mg/kg/day, and wherein the dose is not greater than 60 mg/day to a subject.

Disease States Associated with Spreading Depolarization

In some cases, the subject has been diagnosed with a traumatic brain injury, migraine without aura, migraine with aura, familial hemiplegic migraine, stroke, ischemia, oxygen deprivation, cerebral amyloid angiopathy, chronic subdural hematoma or epilepsy.

In some cases the subject the subject has symptoms associated with migraines (e.g. migraines with aura), and the subject is also diagnosed or suspected to have a demyelination disease, e.g. where the CSD occurrence in a migraine attack promotes neuronal damage, including demyelination. U.S. Provisional Patent Application 63/239,801, which is incorporated herein by reference, describes the treatment of demyelinating diseases with the administration of fenfluramine.

### Fenfluramine

In certain embodiments, fenfluramine is administered, e.g. orally, parenterally, or topically. In particular embodiments, fenfluramine, or a pharmaceutically acceptable salt thereof, is administered orally. In certain embodiments, fenfluramine, or a pharmaceutically acceptable salt thereof, is administered enterally. In some embodiments, fenfluramine, or a pharmaceutically acceptable salt thereof, is administered buccally, sublingually, sublabially, or by inhalation. In other embodiments, fenfluramine, or a pharmaceutically acceptable salt thereof, is administered sublingually. In yet other embodiments, fenfluramine, or a pharmaceutically acceptable salt thereof, is administered parenterally. In particular embodiments, fenfluramine, or a pharmaceutically acceptable salt thereof, is administered intra-arterially, intravenously, intraventricularly, intramuscularly, subcutaneously, intraspinally, intraorbitally, intracranially or intrathecally.

In some embodiments, the fenfluramine or pharmaceutically acceptable salt thereof is administered at a dose of about 0.5 mg/kg/day to about 5 mg/kg/day. In some examples, fenfluramine or a pharmaceutically acceptable salt thereof is administered at a dose oral dose of fenfluramine or a pharmaceutically acceptable salt thereof to the patient in an amount in a range of 0.2 mg/kg/day to 0.8 mg/kg/day up to a maximum of 30 mg/day In some embodiments, the fenfluramine or pharmaceutically acceptable salt thereof is administered daily.

In particular embodiments, the compound is administered to the subject once daily, twice daily, three times daily, once every two days, once weekly, twice weekly, three times weekly, once biweekly, once monthly, or once bimonthly. In certain embodiments, the compound is administered to the subject once daily. In other embodiments, the effective amount in an oral dose of fenfluramine or a pharmaceutically acceptable salt thereof to the patient is an amount in a range of 0.2 mg/kg/day to 0.8 mg/kg/day up to a maximum of 30 mg/day.

In some embodiments, the methods of the present disclosure involve administering a unit dosage form containing from an amount in a range of 0.2 mg/kg/day to 0.8 mg/kg/day up to a maximum of 30 mg/day fenfluramine, or a pharmaceutically acceptable salt thereof, once, twice or three times per day. In some embodiments, the methods of the present disclosure involve administering a unit dosage form containing of fenfluramine, or a pharmaceutically acceptable salt thereof, once, twice or three times per day. In other embodiments, the methods of the present disclosure involve administering a unit dosage form containing from of fenfluramine, or a pharmaceutically acceptable salt thereof, once, twice or three times per day. In particular embodiments, the methods of the present disclosure involve administering a unit dosage form containing from of fenfluramine, or a pharmaceutically acceptable salt thereof, once, twice or three times per day. In yet other embodiments, the methods of the present disclosure involve administering a unit dosage form containing from of fenfluramine, or a pharmaceutically acceptable salt thereof, once, twice or three times per day. In still other embodiments, the methods of the present disclosure involve administering a unit dosage form containing of fenfluramine, or a pharmaceutically acceptable salt thereof, once, twice or three times per day.

Administration of fenfluramine and pharmaceutically acceptable salts thereof is further discussed in the section below.

Administration of Fenfluramine or Pharmaceutical Compositions Thereof

Fenfluramine and pharmaceutically acceptable salts thereof can be administered according to any suitable route of administration for the treatment of a disease or condition associated with spreading depolarization in the brain. For example, standard routes of administration include oral, parenteral, or topical routes of administration. In particular, the route of administration of fenfluramine or a pharmaceutically acceptable salt thereof may be oral (e.g., enteral, buccal, sublingual, sublabial, or by inhalation). Parenteral route of administration of fenfluramine, or a pharmaceutical composition thereof, may be, e.g., intra-arterial, intravenous, intraventricular, intramuscular, subcutaneous, intraspinal, intraorbital, or intracranial. Topical route of administration may be, e.g., cutaneous, intranasal, or ophthalmic.

Pharmaceutical compositions comprising fenfluramine have been described in the art (see, e.g., U.S. Pat. No. 5,883,294, which is herein incorporated by reference).

Fenfluramine and pharmaceutically acceptable salts thereof that are to be administered orally can be formulated as liquids, for example syrups, suspensions or emulsions, or as tablets, capsules or lozenges.

A liquid composition will generally include a suspension or solution of fenfluramine or pharmaceutically acceptable salt in a suitable liquid carrier, for example ethanol, glycerin, sorbitol, non-aqueous solvent such as polyethylene glycol, oils or water, with a suspending agent, preservative, surfactant, wetting agent, flavoring or coloring agent. Alternatively, a liquid formulation can be prepared from a reconstitutable powder, i.e. a liquid which is reconstituted by adding water to a powder.

In some cases, a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example liquid paraffin or fractionated coconut oil can be prepared and filled into a soft gelatin capsule. Fenfluramine and pharmaceutically acceptable salts thereof to be administered parenterally can be formulated, for example, for intramuscular or intravenous administration.

In some instances, a composition for intramuscular administration contains a suspension or solution of active ingredient in an oil, for example arachis oil or sesame oil. A composition for intravenous administration can include a sterile isotonic aqueous solution containing, for example active ingredient, dextrose, sodium chloride, a co-solvent, for example polyethylene glycol and, optionally, a chelating agent, for example ethylenediamine tetracetic acid and an anti-oxidant, for example, sodium metabisulphite. Alternatively, the solution can be freeze dried and then reconstituted with a suitable solvent just prior to administration.

Fenfluramine and pharmaceutically acceptable salts thereof for rectal administration can be formulated as suppositories. A typical suppository formulation will generally include active ingredient with a binding and/or lubricating agent such as a gelatin or cocoa butter or other low melting vegetable or synthetic wax or fat.

Fenfluramine and pharmaceutically acceptable salts thereof to be administered topically can be formulated as transdermal compositions. Such compositions include, for example, a backing, active compound reservoir, a control membrane, liner and contact adhesive.

Non-limiting examples of formulations for buccal, sublingual, and/or sublabial administration may be found in U.S. Pre-grant Publication No. 2012/0058962, U.S. Pre-grant Publication No. 2013/0225626, U.S. Pre-grant Publication No. 2009/0117054, and U.S. Pat. No. 8,252,329; the disclosure of each of which is incorporated herein by reference.

For buccal, sublingual, or sublabial administration, the compositions may take the form of tablets, lozenges, etc. formulated in a conventional manner, as described for oral dosage forms. In some embodiments, the formulation for buccal, sublingual, or sublabial administration includes one or more of taste masking agents, enhancers, complexing agents, and other described above pharmaceutically acceptable excipients and carriers.

Taste masking agents include, for example, taste receptor blockers, compounds which mask the chalkiness, grittiness, dryness, and/or astringent taste properties of an active compound, compounds which reduce throat catch as well as compounds which add a flavor. A taste receptor blocker used in the formulation of the present disclosure may include Kyron T-134, a glycoprotein extract called miraculin from the fruit of the plant synsepalum dulcifcum, ethyl cellulose, hydroxypropyl methylcellulose, arginine, sodium carbonate, sodium bicarbonate, gustducin blockers and mixtures thereof. Compounds which mask the chalkiness, grittiness, dryness and/or astringent taste properties of an active compound include those of a natural or synthetic fatty type or other flavorant such as cocoa, chocolate (e.g., mint chocolate), cocoa butter, milk fractions, vanillin butter fat, egg or egg white, peppermint oil, wintergreen oil, spearmint oil, and similar oils. Compounds which reduce throat catch include combinations of high and low solubility acids. For example, high solubility acids suitable for use here include amino acids (e.g., alanine, arginine etc.), glutaric, ascorbic, malic, oxalic, tartaric, malonic, acetic, citric acids and mixtures thereof. Low solubility acids suitable for use include oleic, stearic and aspartic acids plus certain amino acids such as glutamic acid, glutamine, histidine, isoleucine, leucine, methionine, phenylalanine, serine, tryptophan, tyrosine, valine and fumaric acid. Actual amounts used will vary depending on the amount of throat catch or burn exhibited by the active used but will generally be in the range of 1 to 40%. Flavoring agents include sweeteners and flavors. Examples of suitable sweeteners and flavors include mannitol, sorbitol, maltitol, lactitol, isomaltitol, erythritol, xylitol, sucrose, ammonium glycyrrhizinate, mango aroma, black cherry aroma, sodium citrate, colloidal silicon dioxide, sucralose; zinc gluconate; ethyl maltitol; glycine; acesulfame-K; aspartame; saccharin; acesulfam K, neohesperidin DC, thaumatin, stevioside, fructose; xylitol; honey; honey extracts; corn syrup, golden syrup, misri, spray dried licorice root; glycerrhizine; dextrose; sodium gluconate; stevia powder; glucono delta-lactone; ethyl vanillin; vanillin; normal and high-potency sweeteners or syrups or salts thereof and mixtures thereof. Other examples of appropriate flavoring agents include coffee extract, mint; lamiacea extracts; citrus extracts; almond oil; babassu oil; borage oil; blackcurrant seed oil; canola oil; castor oil; coconut oil; corn oil; cottonseed oil; evening primrose oil; grape seed oil; groundnut oil; mustard seed oil; olive oil; palm oil; palm kernel oil; peanut oil; grapeseed oil; sunflower oil; sesame oil; shark liver oil; soybean oil; hydrogenated castor oil; hydrogenated coconut oil; hydrogenated palm oil; hydrogenated soybean oil; hydrogenated vegetable oil; hydrogenated cottonseed and castor oil; partially hydrogenated soybean oil; soy oil; glyceryl tricaproate; glyceryl tricaprylate; glyceryl tricaprate; glyceryl triundecanoate; glyceryl trilaurate; glyceryl trioleate; glyceryl trilinoleate; glyceryl trilinolenate; glyceryl tricaprylate/caprate; glyceryl tricaprylate/caprate/laurate; glyceryl tricaprylate/caprate/linoleate; glyceryl tricaprylate/caprate/stearate; saturated polyglycolized glycerides; linoleic glycerides; caprylic/capric glycerides; modified triglycerides; fractionated triglycerides; safrole, citric acid, d-limonene, malic acid, and phosphoric acid or salts and/or mixtures thereof.

Enhancers are the agents that increase membrane permeability and/or increase the solubility of a particular active compound. Both issues can be pivotal to the properties of the formulation. An enhancer may be a chelator, a surfactant, a membrane-disrupting compound, a fatty or other acid; a non-surfactant, such as an unsaturated cyclic urea. A chelator may be, e.g., EDTA, citric acid, sodium salicylate, or a methoxysalicylate. A surfactant may be, e.g., sodium lauryl sulphate, polyoxyethylene, POE-9-laurylether, POE-20-cetylether, benzalkonium chloride, 23-lauryl ether, cetylpyridinium chloride, cetyltrimethyl ammonium bromide, or an amphoteric or a cationic surfactant. A membrane-disrupting compound may be, e.g., a powdered alcohol (such as, menthol) or a compound used as lipophilic enhancer. Fatty and other acids include, e.g., oleic acid, capric acid, lauric acid, lauric acid/propylene glycol, methyloleate, lyso-phosphatidylcholine, and phosphatidylcholine. Other enhancers that may be used in buccal, sublingual, and sublabial formulations of the present disclosure include, e.g., lysalbinic acid, glycosaminoglycans, aprotinin, azone, cyclodextrin, dextran sulfate, curcumin, menthol, polysorbate 80, sulfoxides, various alkyl glycosides, chitosan-4-thiobutylamide, chitosan-4-thiobutylamide/GSH, chitosan-cysteine, chitosan-(85% degree N-deacetylation), poly(acrylic acid)-homocysteine, polycarbophil-cysteine, polycarbophil-cysteine/GSH, chitosan-4-thioethylamide/GSH, chitosan-4-thioglycholic acid, hyaluronic acid, propanololhydrochloride, bile salts, sodium glycocholate, sodium deoxycholate, sodium taurocholate, sodium glycodeoxycholate, and sodium taurodeoxycholate.

Buffering materials can be both used to increase solubility and enhance adsorption of active compounds. Examples of suitable buffering materials or antacids suitable for use herein comprise any relatively water soluble antacid acceptable to the Food & Drug Administration, such as aluminum carbonate, aluminum hydroxide (or as aluminum hydroxide-hexitol stabilized polymer, aluminum hydroxide-magnesium hydroxide co-dried gel, aluminum hydroxide-magnesium trisilicate codried gel, aluminum hydroxide-sucrose powder hydrated), aluminum phosphate, aluminum hydroxyl carbonate, dihydroxyaluminum sodium carbonate, aluminum magnesium glycinate, dihydroxyaluminum aminoacetate, dihydroxyaluminum aminoacetic acid, bismuth aluminate, bismuth carbonate, bismuth subcarbonate, bismuth subgallate, bismuth subnitrate, calcium carbonate, calcium phosphate, hydrated magnesium aluminate activated sulfate, magnesium aluminate, magnesium aluminosilicates, magnesium carbonate, magnesium glycinate, magnesium hydroxide, magnesium oxide, and magnesium trisilicate, and/or mixtures thereof. Preferred buffering materials or antacids include aluminum hydroxide, calcium carbonate, magnesium carbonate and mixtures thereof, as well as magnesiumhydroxide. Many of these compounds have the advantage of also being taste masking agents particularly useful for addressing throat catch.

The selection of the other excipients, such as permeation enhancers, disintegrants, masking agents, binders, flavors, sweeteners and taste-masking agents, is specifically matched to the active depending on the predetermined pharmacokinetic profile and/or organoleptic outcome

Liquid drug formulations suitable for use with nebulizers and liquid spray devices and electrohydrodynamic (EHD) aerosol devices will typically include fenfluramine or a pharmaceutically acceptable salt thereof with a pharmaceutically acceptable carrier. Preferably, the pharmaceutically acceptable carrier is a liquid, e.g., alcohol, water, polyethylene glycol, or a perfluorocarbon. Optionally, another material may be added to alter the aerosol properties of the solution or suspension. Desirably, this material is liquid, e.g., an alcohol, glycol, polyglycol, or a fatty acid. Other methods of formulating liquid drug solutions or suspension suitable for use in aerosol devices are known to those of skill in the art (see, e.g., U.S. Pat. Nos. 5,112,598 and 5,556,611, each of which is herein incorporated by reference).

The dose and dosing schedule for administration of fenfluramine (or a pharmaceutically acceptable salt thereof) can vary and is determined in part by the severity of the disease, and the age, weight and general health of the patient. In some embodiments, the composition is administered daily. In other embodiments the composition is administered more than once a day, such as twice a day, three time a day or four times a day. In yet other embodiments, the composition is administered less than once a day, such as every other day, every three days or once a week.

In some embodiments of the methods, the dose of fenfluramine (or a pharmaceutically acceptable salt thereof) may be about 0.1 mg/kg/day to about 3 mg/kg/day. (e.g., twice daily, once daily, twice weekly, or once weekly the dose of fenfluramine (or a pharmaceutically acceptable salt thereof) or may be about 0.1 mg/kg/day to about 2 mg/kg/day. (e.g., twice daily, once daily, twice weekly, or once weekly). In some embodiments, the dose of fenfluramine (or a pharmaceutically acceptable salt thereof) is capped at no more the 60 mg/day. In particular examples, the dose of fenfluramine (or a pharmaceutically acceptable salt thereof) is about (e.g., twice daily, once daily, twice weekly, or once weekly).

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); nt, nucleotide(s); and the like.

In brain slice studies normoxic SD is induced by briefly raising [K⁺]o. An ischemic version of SD, the anoxic depolarization (AD), is induced by removing O2/glucose from the aCSF for about 10 min. Intrinsic optical signals (IOSs) represent change in the way tissue scatters light. Light transmittance (LT), essentially unscattered light, is imaged using a charge-coupled device (CCD) to measure second-by-second regional DLT during periods of 1 h or more. The front of the propagating SD or AD event is imaged as an elevated LT, caused by cell swelling. A negative voltage shift, the electrophysiological signature of SD, is simultaneously recorded extracellularly. Normoxic SD is typically the cortical occurrence in migraine, while an anoxic condition is more typical of ischemic attacks and stroke. The Figures and Example below demonstrated that sigma-1 agonists and positive modulators were effective in reducing SD in mouse brain slices and that fenfluramine has a positive effect on SD under anoxic conditions (AD).

### Example 1: Measurement of Spreading Depolarization in Mouse Brain

To determine the threshold for evoked spreading depolarization in mouse brain slices, mice (C57Bl6J, P30-50) were deeply anesthetized by ketamine/xylazine mix (i.p. 85 and 15 mg/ml, respectively), cardiac perfused with cutting solution (110 mM N-Methyl-D-glucamine, 10 mM glucose, 6 mM magnesium sulfate, 3 mM KCl, 25 mM sodium bicarbonate, 1.25 mM sodium monophosphate, 0.2 mM calcium chloride, and 0.4 mM ascorbic acid equilibrated with 95% O2/5% CO2), and decapitated. The brain was rapidly extracted, cerebellum removed, forebrain hemisected, and 300 μm thick coronal slices were cut on a vibratome.

Slices were first incubated in the cutting solution at 37 C for 10 minutes and then transferred to artificial cerebrospinal fluid (“ACSF”: 126 mM sodium chloride, 10 mM glucose, 1 mM magnesium sulfate, 3 mM KCl, 25 mM sodium bicarbonate, 1.25 mM sodium monophosphate, 2 mM calcium chloride, and 0.4 mM ascorbic acid equilibrated with 95% O2/5% CO2).

In all in vitro experiments, a pair of slices were then transferred to a recording chamber continuously superfused with ACSF at 2.5-3.0 mm/minute 33-34° C. For pharmacological analysis, slices were pre-incubated in the recording chamber with test compound for at least for 20 minutes. Slow tissue depolarizations (SD) were detected by DC potential shift or imaging of intrinsic optical signals (IOS) in the brain slice. DC potential were recorded using glass pipette electrodes containing ACSF (2-3MO). Signals were amplified with a DC amplifier (MultiClamp 700B), digitized (Digidata 1550B) and analyzed with the Clampfit program (Molecular instruments). IOS were detected by acquiring images of brain slices with a CMOS camera (Hamamatsu ORCA). Images were acquired at 0.2-0.5 Hz and analyzed with imageJ software.

Briefly, intrinsic optical signal imaging is similar to fMRI in that it relies upon measuring areas of oxygenated vs deoxygenated blood to construe activity, but rather than using an MRI machine it uses light and a scientific camera. ISOI takes advantage of the spectral properties of hemoglobin, which has different absorption properties when oxygenated or deoxygenated. The basic principle of ISOI is that when brain tissue is directly illuminated, the active areas reflect less light than non-active areas. The more active a brain area the less light reflected, making highly active areas appear darker. There are three main reasons why neuronal tissues change their optical properties when active: active neurons scatter more light (due to swelling and ion movements), natural fluorophores within the cells react (including hemoglobin) and the changes in blood volume/oxidation. See also, Hillman EMC. (2007) Optical brain imaging in vivo: techniques and applications from animal to man. J Biomed Opt. 12(5): 051402. doi: 10.1117/1.2789693

SD waves were induced in two different assays.

First, by incrementally elevating the KCl concentration of bath ACSF every 5 minutes (initial starting concentration of 6 mM, then elevated by +1 mM at 5-minute intervals). The KCl concentration that triggered the first SD was considered as the SD threshold. In experiments using the test drug (fenfluramine), the drug was added to the solution before the KCl challenge.

The second assay of SD entailed reducing the oxygen and glucose levels in the bath solution. OGD-SD was induced by incubating cortical slices in modified ACSF that were equilibrated with 95% N2/5% CO2 and glucose concentration reduced to 0 or 2 mM and substituting with sucrose to maintain osmolarity.

Test drugs (including fenfluramine) were present in the bath solution throughout the OGD challenge. The latency to SD onset in the brain slice was used to evaluate SD threshold.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

What is claimed is:

1. A method of inhibiting spreading depolarization in a subject, the method comprising:
administering a therapeutically effective amount of a sigma-1 agonist or a sigma-1 positive modulator to the subject,
thereby inhibiting the spreading depolarization.

2. The method of claim 1, wherein the subject is administered a sigma-1 agonist.

3. The method of claim 2, wherein the sigma-1 agonist is selected from the group consisting of PRE-084, blarcamesine, donepezil, fluvoxamine, citalopram, amitriptyline, L-687,384, cutamesine, dextromethorphan, N,N-dimethyltryptamine, pentazocine, opipramol and DTG.

4. The method of claim 1, wherein the subject is administered a sigma-1 positive modulator.

5. The method of claim 4, wherein the sigma-1 positive modulator is selected from the group consisting of SKF83959; SKF38393; SCH23390; fenfluramine, methylphenylpiracetam, E1R, Anavex 2-73, and OZP002.

6. The method of claim 5, wherein the sigma-1 positive modulator is fenfluramine.

7. The method of claim 5, wherein the sigma-1 positive modulator is not fenfluramine.

8. The method of claim 1, wherein the sigma-1 agonist or a sigma-1 positive modulator is administered prophylactically.

9. The method of claim 1, further comprising diagnosing the subject with spreading depolarization or an elevated risk of spreading depolarization before the administering.

10. The method of claim 9, wherein the diagnosing comprises performing an electroencephalography (EEG) measurement of the subject.

11. The method of claim 10, wherein the diagnosing comprises performing a first EEG measurement of the subject, administering a test amount of a sigma-1 agonist or a sigma-1 positive modulator to the subject, and performing a second EEG measurement of the subject.

12. The method of claim 9, wherein the administering is performed between 30 seconds and 60 minutes after the diagnosis.

13. The method of claim 1, wherein the subject has been diagnosed with traumatic brain injury, migraine without aura, migraine with aura, familial hemiplegic migraine, stroke, ischemia, oxygen deprivation, cerebral amyloid angiopathy, chronic subdural hematoma, epilepsy, or a combination thereof.

14. A method of treating a subject with a disease associated with spreading depolarization, the method comprising:
administering a therapeutically effective amount of a sigma-1 agonist or a sigma-1 positive modulator to the subject,
thereby alleviating or preventing symptoms or morbidity of the disease.

15. The method of claim 14, wherein the disease is selected from the group consisting of traumatic brain injury, migraine without aura, migraine with aura, familial hemiplegic migraine, stroke, ischemia, oxygen deprivation, cerebral amyloid angiopathy, chronic subdural hematoma, epilepsy, or a combination thereof.
